(19) United States
(12) Patent Application Publication
(10) Pub. No.: US 2004/0225340 A1
(43) Pub. Date: Nov. 11, 2004

(54) Light/breath/meditation device

(76) Inventor: James W. Evans, Ransomville, NY (US)

Correspondence Address: National IP Rights Center, LLC, Suite 400, 550 Township Line Road, Blue Bell, PA, 19422, US

(21) Appl. No.: 10/797,914

(22) Filed: Mar. 10, 2004

Related U.S. Application Data
(60) Provisional application No. 60/452,712, filed on Mar. 10, 2003.

(51) Int. Cl. A61N 1/00

(52) U.S. Cl. 607/88

(57) ABSTRACT

A device to irradiate a user's eyes comprising: a translucent shield in the form of glasses applied over a user's eyes, said shield blocking the portion of ambient light outside the blue-green color spectrum; a LED source of dim intensity colored light within the shield which irradiates the eye with diffused light; and means for oscillating the LED source to alert the user when to inhale and when to exhale, and/or meditate.

### CLAIM OF PRIORITY

This application claims the priority of Provisional Application No. U.S. Ser. No. 60/452,712 entitled Prayer and Meditation Aid, filed Mar. 10, 2003.

### FIELD OF THE INVENTION

The present invention is directed to a light emitting device and method for irradiating the wearer's eyes and signaling the wearer when to inhale and exhale for slow breathing, and/or to facilitate meditation.

### BACKGROUND OF THE INVENTION

The role of light in human health has been shown to be very significant. Light is the primary stimulus in the regulation of neurochemicals, hormones, chronobiological (circadian) rhythms, and seasonal cycles. The full spectrum and various colors of light are used in the treatment of medical and psychiatric illness: pain, seasonal and non-seasonal depression, insomnia, anxiety, and other chronobiological disorders. Light therapy has long been used in the specific treatment of Seasonal Adjustment Disorder, (SAD), a type of depression.

The intensity of light in research studies on SAD and other chronobiological disorders ranges from very bright (10,000 lux) to very dim (250 lux or less). The exact mechanism of light therapy continues to be researched. Current theories involve the role of light in: (1) phase shifting chronobiological rhythms; and (2) neurochemical production and regulation in the human body (specifically melatonin). Current research has demonstrated that the blue-green end of the color spectrum is the most potent wavelength region for shifting circadian rhythms and regulating melatonin production. Light is used therapeutically to irradiate the eyes and other areas of the body.

A number of articles have been published in this area of SAD and chronobiological Disorders. These include: Depression Guideline Panel, 1993. AHCPR Publication No. 93-0551, U.S.G.P.O., Washington D.C.; Rosenthal, N. E., 1995. Light Therapy. In: Gabbard, G. O. (Ed.), Treatment of Psychiatric Disorders. American Psychiatric Press, Washington D.C., pp. 1263-1273; Brainard, G. C.; Hanifin, J. P.; et al. Action Spectrum for Melatonin Regulation in Humans: Evidence for a Novel Circadian Photoreceptor. Journal of Neuroscience, Aug. 15, 2001, 21(16): 6405-6412; Benedetti, F.; Columbo, C.; et al. Morning Light Treatment Hastens the Antidepressant Effect of Citalopram: A Placebo-Controlled Trial. Journal Clinical Psychiatry; 2003; 64; 648-653; Wright, H. and Lack, L. Effects of Light Wavelength on Suppression and Phase Delay of the Melatonin Rhythm. Chronobiology International, 2001, 18(5), 801-808; Lambert, G. W.; Reid, C.; et al. Effect of Sunlight and Season on Serotonin Turnover in the Brain. Lancet, 2002, 360 (9348); 1840-1842.

One category of devices involves one or more colored light bulbs in a light fixture, which illuminates the place in which the user is located. One example is the invention in U.S. Pat. No. 5,447,527: Therapeutic Light Method. This lamp emits green light for treating SAD or other chronobiological disorders. The lamp is in a fixed position and the user positions himself/herself in the path of the light. The lamp is simple to operate and the user can perform activities while being irradiated. However, the user must remain within 30 inches of the light, since the device is positioned on a table. U.S. Publish Application No. 2001/0056293, Brainard discloses a blue light system for stimulating or regulating neuroendocrine, circadian, and photoneural systems in mammals based upon the discovery of peak sensitivity ranging from 425-505 nm; a light meter system for quantifying light which stimulates or regulates mammalian circadian, photoneural, and neuroendocrine systems. The invention also relates to translucent and transparent materials, and lamps or other light sources with or without filters capable of stimulating or regulating neuroendocrine, circadian, and photoneural systems in mammals, treatment of light responsive disorders, eating disorders, menstrual cycle disorders, non-specific alerting and performance deficits, hormone-sensitive cancers, and cardiovascular disorders. The green and blue light devices do not address the issue of slow breathing.

Another category of devices positions the lights on or around the head of the user. Examples of this category are in the U.S. Pat. No. 4,858,609: Bright Light Mask; and in U.S. Pat. No. 6,092,906: Personal/Private Light Screen, and in U.S. Pat. No. 5,242,376: Relaxation Device and Method.

U.S. Pat. No. 4,858,609 discloses a bright light mask system for shining a high intensity light into a subject's eyes at preselected time periods to modify circadian rhythms. The system includes a mask adapted to be worn by the subject for covering the subject's eyes regardless of body position. The mask includes at least one light admitting aperture that is transparent to light energy. A light source is coupled to the aperture for generating and directing light into the subject's eyes. A light intensity of at least 2000 lux of light having a wavelength in the range of 500 to 600 nanometers is delivered to each of the subject's eyes. A controller dictates the intensity of the light generated and the timing during which the light is on.

U.S. Pat. No. 5,242,376 discloses a light emitting relaxation device and method for placing a subject in a relaxed state. The relaxation device includes a portable mask that has a flashing light source. When the mask is positioned on the subject's head, a shade of violet reflected light is emitted into the subject's eyes. The mask, preferably, is totally self-contained having built-in controls to set the speed, intensity, and duration of the flashing light.

U.S. Pat. No. 6,092,906 discloses a lightweight light screen that emits a low-intensity, diffused light of uniform brightness from its interior surface. The light screen covers the user's eyes when worn, thereby preventing ambient light from entering the user's eyes. The wearer of the light screen can see only the emitted light. The nature of the emitted light in combination with isolation from ambient visual stimuli provided a restful and therapeutic environment for the wearer. The light screen has two apertures with shades that can be used to uncover the apertures. The apertures are positioned with respect to each eye such that when uncovered the wearer can read or perform other short-focal-distance tasks that require eyesight while obtaining benefits from the emitted light. The combined area of the apertures is less than 5% of the light emitting area of the light screen so the user can maintain his/her sense of isolation while performing tasks and continue to derive benefits from irradiation.

These devices are used for treating psychiatric conditions such as SAD, for meditation, and relaxation. However, in all the previous devices the visual field is fully or partially obstructed. Thus the wearer is not able to be mobile during use. Also, none of these devices address the issue of slow breathing.

U.S. Pat. No. 5,274,403 discloses a system including lenses which transmits light wavelengths in the 400-600 nanometer range to maximize the inhibition of melatonin secretion. This device also does not address the issue of slow breathing. There is a category of meditation devices that do not primarily focus on the application of light and breathing. Examples of these are U.S. Pat. Nos. 4,167,075; 6,205,589; and 4,330,889; and WIPO Patent Numbers: WO 02028464; and WO 2003082056.

U.S. Pat. No. 4,167,075, for example, discloses a meditation aid, which has a rigid frame whose periphery defines a geometric shape. A plurality of identical flexible means for suspending the frame are attached to it and joined at a single point. The suspending means and frame define a three dimensional geometric figure such as a cone, pyramid or the like. Means are provided for adjusting the distance from the joinder point to the frame along with means for hanging the flexible means from a superstructure. Further means are provided for raising and lowering the frame.

U.S. Pat. No. 6,205,589 discloses a meditation enhancing apparatus, which is made of a highly conductive metal such as copper. The head covering or helmet has several ports which are adapted to hold crystals, the ports being connected to conduits. The conduits serve to hold the crystals in position directly in front of the user's eyes and temples, as well as to conduct electromagnetic and other energy to the user's brain. The user may select crystals which have been effective in enhancing previous meditation sessions for placement in the device.

U.S. Pat. No. 4,330,889 discloses a sleeping and meditation bag. The device has a first position to accommodate the user in a sitting, cross-legged posture and a second position to accommodate the user in a reclining posture. The bag comprises a first portion to accommodate the shoulders and extended elbows of the user in the first position. A second portion of the bag accommodates the trunk of the user and is joined to the first portion. This second portion has a width substantially coincident to the greatest width of the first portion. A third portion has one end adjacent the second portion and is substantially the same width as the second portion to accommodate the extended knees of the user in the first position. The third portion is tapered towards an end surface of the bag to accommodate the feet of the user in the second position.

WIPO Patent No. W002028464 discloses a relaxation device for assisting a user in relaxing the mind and body, and usefully producing a relaxation effect such as sleep induction, meditation assistance, and blood circulation acceleration. The device includes an earth line, an electrode connected to the earth line and in contact with the body, and a direct current power supply connected in serial to the electrode, for applying a negative voltage of −12V or −27V to the electrode. By earthing the body, static electricity stored in the body is removed, and by applying a predetermined negative voltage to the body, relaxation is easily attained.

WIPO Patent No. WO2003082056 discloses an apparatus for assisting meditation. The apparatus comprises a turntable mounted on a central drive shaft, the central drive shaft having a seat mounted thereon. A drive means is connected to the central drive shaft in order to rotate the central drive shaft and the turntable. A speed control means is connected to the drive means to control the rotational speed of the central drive shaft wherein the speed control means is located in a position in which the speed control means may be adjusted by a user sitting on the seat

The field of mediation involves the use of various breathing patterns and repetitions of a mantra, prayer, verses, etcetera to achieve a meditative state. One yoga exercise is known as “belly” or “abdominal” breathing. Another adaptation of this breathing technique is called “focused” breathing. Recent research in medicine has determined that breathing at the specific rate of six breath cycles per minute has very significant cardiac, respiratory, and other physical and emotional health benefits. This breathing rate is designated “slow breathing”. To achieve these benefits, the user does not need to combine any religious or philosophical practices (prayer, mantra, etc.) with the slow breathing.

The field of study on slow breathing (approximately six breath cycles per minute) demonstrates the enhancement of heart rate variability and baroreflex sensitivity by synchronizing inherent cardiovascular rhythms. The reduction in the responses of these heart rhythms is of considerable clinical importance because they are “an independent predictor of increased future risk after a recent heart attack, or in heart failure.” Studies on slow breathing have demonstrated that when a human breathes at the approximate rate of one breath cycle every 10 seconds (inhale for 5 seconds and exhale for 5 seconds), that a number of biological functions improve, including cardiovascular and respiratory function, increased respiratory sinus arrhythmia, the arterial baroreflex, oxygenation of the blood, and exercise tolerance. In the case of chronic heart failure, slow breathing reduces the exaggerated sensitivity of the respiratory chemoreflex, and improves irregular breathing. “Slow respiration may reduce the deleterious effects of myocardial ischaemia, and, in addition, it increases calmness and well-being. These effects result from, at least in part, synchronization of respiratory and cardiovascular central rhythms. A respiratory rate of around 6/min coincides with and thus augments the 10 second (6/min) Mayer waves, and so increases the power of vagal respiratory sinus arrhythmia. The favorable effects of slowed breathing may be mediated, at least partly, by a modulation of autonomic activity at both central and peripheral (baroreflex) levels.” Bernardi, L.; Sleight, P.; et al. Effects of Rosary Prayer and Yoga Mantras on Autonomic Cardiovascular Rhythms: Comparative Study. BMJ, 2001; 323: 1446-1449.

U.S. Pat. No. 6,212,135 illustrates an apparatus for assistive breathing. The system disclosed has a globe shaped cuing means that sits atop a control structure. This device does not allow the user to be mobile, and also teaches complex breathing rhythms which might be difficult for some people to achieve. For example, one pattern of breathing decreases the number of respiratory cycles per minute to one or two. This invention is not designed as a light therapy irradiation device.

Other devices for assisting with certain breathing patterns are U.S. Pat. Nos. 4,711,585; 4,493,043; and 4,491,423. These inventions do not specifically pace breathing at six breath cycles per minute and are not designed as a light therapy irradiation device.

While there are a number of prior art devices and systems which facilitate light irradiation or assistive breathing, none have combined the benefits of light irradiation with those of slow breathing at approximately 6 breath cycle per minute.

There is a long felt need for a device, system, and method which would enable a person to combine the best features of light irradiation with those of slow breathing. Such a device would provide users with the beneficial aspects of both.

It is a principal object of the present invention to provide a device which supplies light in the green-blue wavelength with timed lighting for signaling slow breathing at approximately 6 breaths per minute.

It is a further object of the present invention to provide a device which filters light and allows transmission of the blue-green spectrum while simultaneously assisting the user in signaling the timing of breathing for health benefits.

It is a further object of the present invention to provide a portable device which may take the form of glasses or goggles and which assist in transmitting blue-green light and further assists in signaling slow respiration.

These and other objects of the present invention will become apparent from the summary and detailed description of the present invention.

### SUMMARY OF THE INVENTION

The present invention is directed, in a broad embodiment, to a colored translucent shield, which fits over a user's eyes and which enables the wearer to benefit from light, preferably in the blue-green spectrum. Further, a light source inside the shield is then pulsed on and off to signal the wearer to slowly inhale and exhale, thus providing the benefits of slow breathing. The device may also be used as a meditation aide, utilizing colored light, breath focus, and rhythmic pacing of meditative words, or verses. The device is comfortable, portable, and does not obstruct the visual field; thus allowing the wearer to be mobile while using the device. The light intensity is dim; thus avoiding potential side effects from high intensity light irradiation.

In a most preferred embodiment, light passes through the translucent shield to the wearer's eyes. The shield may comprise a pair of glasses or goggles and have a light source mounted on the interior. The shields block a portion of ambient light, thus allowing the transmission of light in the blue-green spectrum. The light source comprises light emitting diodes (LEDs). In a preferred embodiment, one LED per eye irradiates the eye with very dim intensity and diffused light. The LEDs are controlled by a battery powered controller, which causes the LEDs to pulse on and off.

The pulsing LEDs signal the wearer to inhale (e.g., when the LED is on for 5 seconds), and exhale (e.g., when LED dims off for 5 seconds) and/or to recite a meditation word, verse, or prayer. An electronic timing control circuit is mounted in a small control box having an on/off switch. This control box is connected to the LEDs with a power cord.

The box easily fits in the wearer's pocket or belt, thus allowing mobility. The control box has space for mounting a meditation word, prayer, verse, or picture. The colored light emitting shield when worn by the meditator will create the visual perception of colored light in the environment and thus facilitate meditation.

In accordance with the present invention, a method for providing light and to signal breathing rate comprising the following steps: irradiating a user's eye through a translucent colored shield to filter out all light except light in the blue-green spectrum; and irradiating the user's eye with light pluses timed to alert the user when to inhale and when to exhale; and/or meditate on a mantra or verse.

In a further embodiment, the invention is a device to irradiate the wearer's eyes comprising: a translucent shield applied over a user's eyes to block a portion of ambient light outside the blue-green color spectrum; a source of dim intensity colored light within the shield which irradiates the eye with diffused light and which oscillates to alert the user when to inhale and when to exhale.

In still a further embodiment, the invention is a device to irradiate a user's eyes comprising: a translucent shield in the form of glasses applied over a user's eyes, said shield blocking the portion of ambient light outside the blue-green color spectrum; a LED source of dim intensity colored light within the shield which irradiates the eye with diffused light; and means for oscillating the LED source to alert the user when to inhale and when to exhale.

### DESCRIPTION OF THE FIGURES

FIGS. 1 is a frontal perspective view of the novel device of the present invention.

FIG. 1a is a rear perspective view of the novel device of the present invention.

FIG. 2 is an alternative embodiment of the present invention.

FIG. 3 is a further embodiment of the invention.

### DETAILED DESCRIPTION OF THE PREFERRED EMBODIMENT

The present invention is described in reference to the enclosed figures wherein the same numbers are utilized where applicable. In a broad embodiment, the invention comprises a colored light transmitting shield, which enables the wearer to benefit from irradiation in the blue-green spectrum. In a preferred embodiment the shield may comprise a pair of glasses or goggles 10.

An internal light situated within the glasses/goggles, is gently pulsed on and off to signal the wearer to slowly inhale and exhale, thus providing benefits from a slow breathing rhythm. The device may also be used in conjunction with a meditation aide, such as a mantra, or prayer utilizing the light/breath focus, and rhythmic pacing for repeating a meditative word, or verse.

The device is comfortable, portable, and does not obstruct the visual field; thus allowing the wearer to be mobile while using the device. The light pulses on and off to signal the wearer to breath at approximately 6 breath cycles per minute. Thus, the wearer is able to benefit from both light irradiation and slow rhythmic breathing.

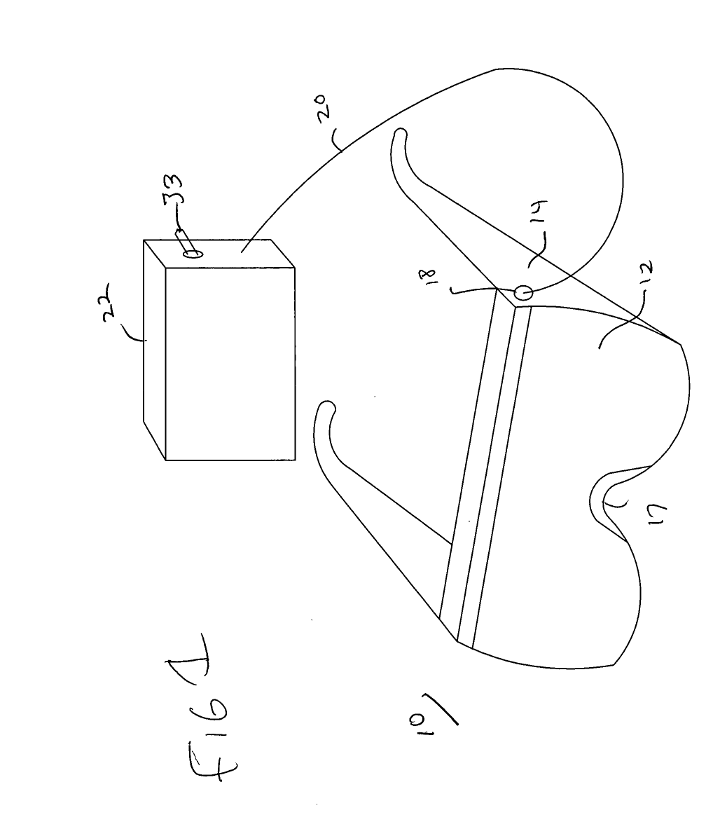

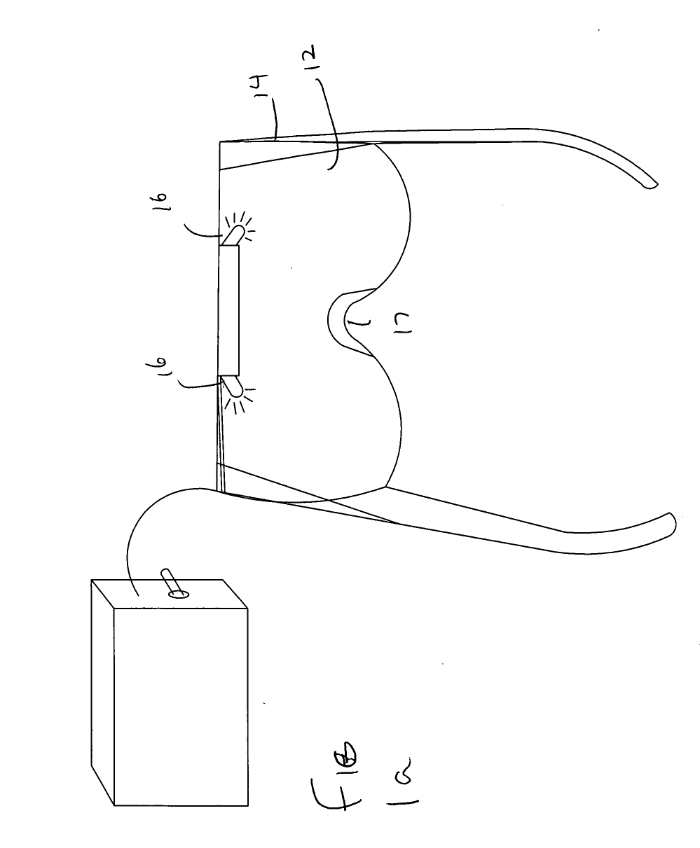

More specifically, referring to FIGS. 1 and 1a, a most preferred embodiment is shown. The invention comprises translucent blue shield glasses or goggles 10 which filter out a portion of ambient light, except that in the blue spectrum. The glasses or goggles 10 have lenses 12 and side flaps 14 which function to fully encase the wearer's visual field. The blue colored, translucent shield 12 filters the ambient light for irradiation benefits.

As shown I FIG. 1a, in a most preferred embodiment, the interior of the glasses/goggles has two green light emitting diodes 16 (LED), each on opposite sides of the bridge 17 of the goggles/glasses, which irradiate each eye with dim and diffused oscillating light. The LEDs 16 are attached via electric connection 18 and wire 20 which attach to a control box 22 which may be placed in the wearer's pocket or on his or her belt.

The control box includes a timer 24 which is set to pulse the LEDs for a period on followed by a period off. In a most preferred embodiment, the system is designed such that the user inhales when the LED pulses on and exhales when it dims off at approximately 6 breath cycles per minute. An on-off switch 33 is also provided. The system has a controller which facilitates the timing of the on-off cycles to signal 6 breath cycles per minute. A control switch 31 can vary the timing. The system operated by an on-off switch 28 and may be powered by batteries. The lights are timed to gradually go on and off to pace the act of respiration. The light source itself is colored in the blue-green range to provide cues of a pleasant color and benefits of irradiation in this color spectrum.

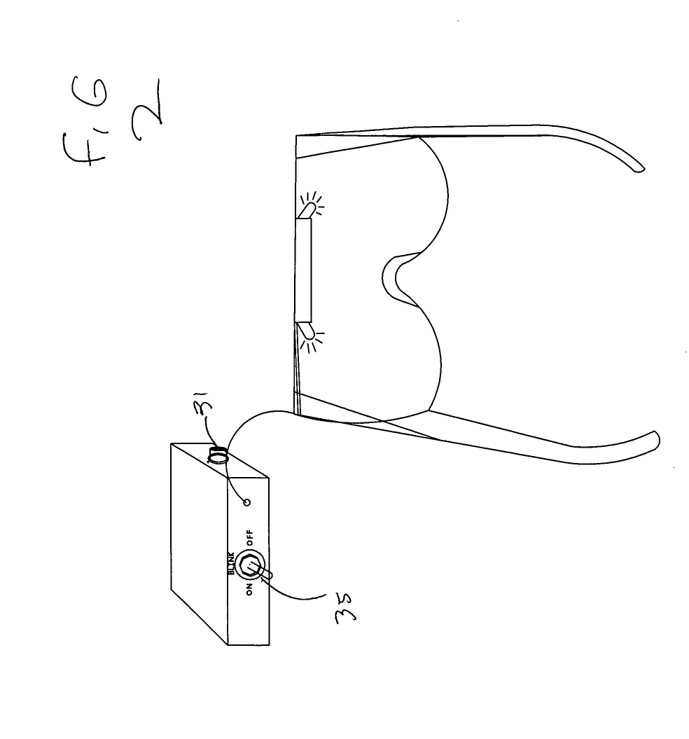

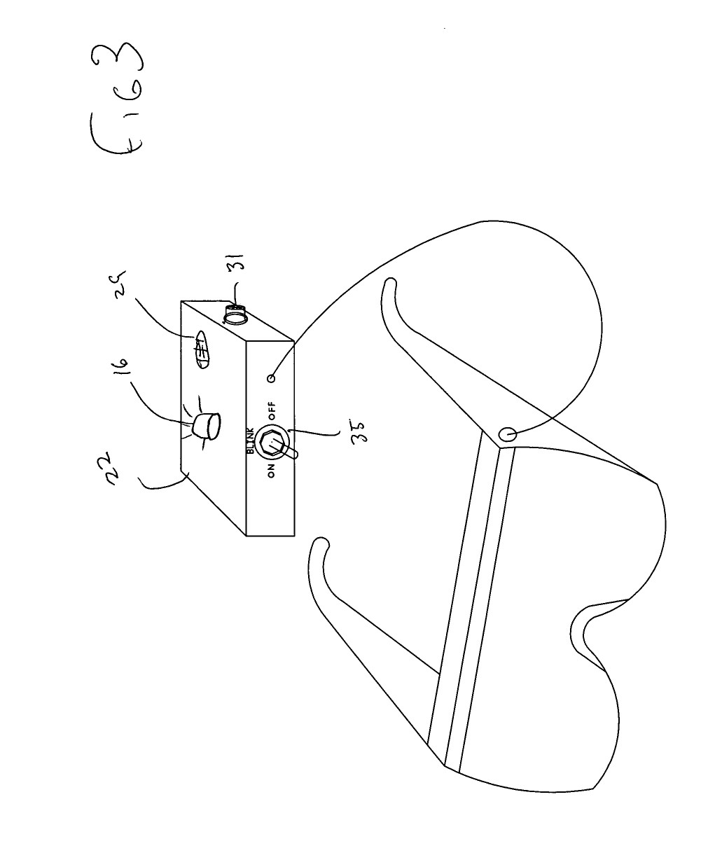

A number of alternative embodiments are suggested by the present invention. For example, as shown in FIG. 3, an embodiment is suggested wherein the LEDs 16 or alternative light sources are mounted on the control box 22 to signal the pace for slow breathing. This would be for people who do not want to wear lights in glasses. The user could direct the light from the control box 22 to irradiate the area he chooses. Shown in FIG. 3, in yet another embodiment, the lights 16 could be mounted on the control box with an audio signal 29 for pacing breathing. An additional switch position would allow the user to turn the lights off and listen to the audio signal for slow breathing. Finally the present invention could be carried out by an embodiment in which the lights remain continuously “on” or go “on and off.” This could be accomplished with a three-way switch 35 as shown in FIGS. 2 and 3.

Finally, the present invention suggests the use of clear lens glasses, or lens having any color with lights that are full spectrum or other monochromatic colors. Full spectrum lights or other monochromatic colored lights are mounted on the control unit. The control unit is miniaturized and mounted on the glasses (no electric cord).

By training with the present invention, it is anticipated that a user can regulate the number of respiratory cycles per minute to approximately six. The critical object is that the present invention is preset to pace slow breathing at the rate researchers have determined to be most beneficial for health and well-being (six breath cycles per minute).

Hence, the present invention in a most preferred embodiment, provides the wearer with the blue-green light for the benefits of irradiation; pulsed rhythmic light to signal the user to pace respiration for the benefits of slow breathing, and may further be used in conjunction with a mantra or verse.

The present invention has been designed with reference to the enclosed Figures and detailed description. It is to be appreciated that the true nature and scope of the present invention is to be determined with reference to the claims appended hereto.

1. A method for providing light and to signal slow breathing irradiating a user's eyes through a translucent colored shield to filter out all light except light in the blue-green spectrum;
irradiating the user's eyes with light pulses timed to alert the user when to inhale and when to exhale;

2. The method of claim 1 wherein the light pulses are in the blue-green spectrum.

3. The method of claim 2 wherein the light pulses are emitted via a light emitting device.

4. A device to irradiate the wearer's eyes comprising:
a translucent shield applied over a user's eyes to block a portion of ambient light outside the blue-green color spectrum;
a source of dim intensity colored light within the shield which irradiates the eye with diffused light and which oscillates to alert the user when to inhale and when to exhale.

5. The device of claim 4 wherein the light comprised of LEDs (light emitting diodes) which signal the wearer to inhale for approximately 5 seconds and exhale for approximately 5 seconds.

6. The device of claim 5 wherein the LEDs are regulated by a controller.

7. This device of claim 6 wherein the control box is connected to the LED by an electronic cord.

8. The device of claim 6 wherein the control box may be affixed to a user's belt.

9. The device of claim 6, wherein the control box has a space for mounting a meditation word, prayer, verse, or picture.

10. A device to irradiate a user's eyes comprising:
a translucent shield in the form of glasses applied over a user's eyes, said shield blocking block the portion of ambient light outside the blue-green color spectrum;
an LED source of dim intensity colored light within the shield which irradiates the eye with diffused light;
and means for oscillating the LED source to alert the user when to inhale and when to exhale, and/or repeat a prayer, mantra, or meaningful verse.
